(19) United States
(12) Patent Application Publication
(10) Pub. No.: US 2008/0076139 A1
(43) Pub. Date: Mar. 27, 2008

(54) Methods and compositions for detecting the activation states of multiple signal transducers in rare circulating cells

(76) Inventor: Sharat Singh, Los Altos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/525,598

(22) Filed: Sep. 21, 2006

(51) Int. Cl. G01N 33/574

(52) U.S. Cl. 435/7.23; 435/7.4

(57) ABSTRACT

Methods and kits for detecting the activation states of a plurality of signal transducers of circulating cells of a solid tumor in a specific, multiplex, high-throughput assay are described. The methods comprise: contacting the signal transducers extracted from the cells with first, second, and third binding partners specific for each of the signal transducers to produce signal transducer-binding partner complexes. The second binding partners bind the corresponding signal transducers independent of their activation state and are labeled with a first moiety, and the third binding partners bind the corresponding signal transducers dependent of their activation state and are labeled with a second moiety. The first and second moieties are detected as an indication of the activation states of the plurality of signal transducers.

### BACKGROUND OF THE INVENTION

The field of the invention is detecting activation states of a plurality of signal transducers of circulating cells of a solid tumor in a specific, multiplex, high-throughput assay.

Tumor cells often are found in the blood of patients with various early stages of cancer, these cells are referred to as “micrometastases” (disseminated tumor cells) and also in metastatic cancers. The number of the tumor cells in blood depends from the stage and type of the tumor. Tumors are extremely heterogeneous, thus a biopsy from a single site might not represent the heterogeneity in a tumor population. Biopsy is typically obtained on primary tumors; most metastatic tumors are not biopsied, making molecular analysis of tumor samples even more difficult. During tumor metastasis, the most aggressive tumor cells leave the primary tumor and travel through the blood and lymphatic system to reach a distant location. Thus, circulating tumor cells from blood represent the most aggressive and homogenous population of tumor cells. The number of metastatic tumor cells in blood can vary from one to several thousand cells per milliliter of blood. Accordingly, specific and sensitive methods are needed to detect these cells for diagnostic and prognostic purposes.

Methods for detecting circulating cancers cells are disclosed in US Pat. Appl. Pub. No. 20040157271 to Kirakossian et al. and U.S. Pat. Appl. Pub. No. 20060008807 to O'Hara et al. Irish et al. (Cell (2004) 118:217-28) describe using multiparameter flow cytometry to monitor phospho-protein responses to environmental cues in acute myeloid leukemia at the single cell level.

### SUMMARY OF THE INVENTION

The invention provides methods for detecting activation states of a plurality of signal transducers of circulating cells of a solid tumor in a specific, multiplex, high-throughput assay. The methods comprise: contacting the signal transducers extracted from the cells with first, second, and third binding partners specific for each of the signal transducers to produce signal transducer-binding partner complexes, wherein the second binding partners bind the corresponding signal transducers independent of their activation state and are labeled with a first moiety, and the third binding partners bind the corresponding signal transducers dependent of their activation state and are labeled with a second moiety. The second and third binding partners are restrained and organized on an array, and the first and second moieties at each distinct region of the array are detected as an indication of the activation states of each of the plurality of signal transducers.

One method comprises the steps of: contacting the signal transducers extracted from the cells with first, second, and third binding partners specific for each of the signal transducers, and a solid support having a surface comprising: (a) the first binding partners restrained in an array and adapted to selectively immobilize and organize the corresponding signal transducers on the array; or (b) a plurality of capture molecules restrained in an array, wherein the first binding partners comprise capture tags that specifically bind the capture molecules, and the capture molecules are adapted to selectively immobilize and organize the corresponding tagged binding partners on the array, whereby the first, second, and third binding partners specifically bind to and restrain the corresponding signal transducers on the array, wherein the second binding partners bind the corresponding signal transducers independent of their activation state and are labeled with a first moiety, the third binding partners bind the corresponding signal transducers dependent of their activation state and are labeled with a second moiety, and the first and second moieties generate first and second signals wherein the second signal is detectable and generated dependent on channeling of the first signal between the moieties; and detecting the generated second signal as an indication of the activation states of the plurality of signal transducers.

Another method comprises the steps of: incubating the signal transducers extracted from the cells in solution with optional first, and with second and third binding partners specific for each of the signal transducers to specifically bind the corresponding signal transducers to the second and third binding partners; contacting the second and third binding partners that have specifically bound to the corresponding signal transducers with a solid support having a surface comprising: (a) the first binding partners specific for each of the signal transducers restrained in an array and adapted to selectively immobilize and organize the corresponding signal transducers on the array; or (b) a plurality of capture molecules restrained in an array, wherein the first or the second and third binding partners comprise capture tags that specifically bind the capture molecules, and the capture molecules are adapted to selectively immobilize and organize the corresponding tagged binding partners on the array, whereby the second and third binding partners are restrained on the support surface, wherein prior to, or concurrently with, being restrained on the support surface the second and third binding partners form signal transducer-binding partner complexes with the corresponding signal transducers and first binding partners, the second binding partners bind the corresponding signal transducers independent of their activation state and are labeled with a first moiety, and the third binding partners bind the corresponding signal transducers dependent of their activation state and are labeled with a second moiety; and detecting the first and second moieties of the restrained second and third binding partners as an indication of the activation states of the plurality of signal transducers.

The invention also provides kits for performing the methods of the invention comprising the first, second, and third binding partners; and the solid support. In one embodiment of the kit, the solid support has a surface comprising: (a) the first binding partners restrained in an array and adapted to selectively immobilize and organize the corresponding signal transducers on the array; or (b) a plurality of capture molecules restrained in an array, wherein the first binding partners comprise capture tags that specifically bind the capture molecules, and the capture molecules are adapted to selectively immobilize and organize the corresponding tagged binding partners on the array; and the first and second moieties are adapted to generate first and second signals wherein the second signal is detectable and generated dependent on channeling of the first signal between the moieties. In another embodiment of the kit, the solid support surface comprises capture molecules restrained in the array, wherein the first binding partners or second and third binding partners comprise capture tags that specifically bind the capture molecules to restrain and organize the binding partners in the array.

### BRIEF DESCRIPTION OF THE DRAWINGS

FIG. 1 depicts three binding partners specifically bound to an activated signal transducer.

FIG. 2 depicts an assay scheme where labeled binding partners that have specifically bound to an activated signal transducer are restrained on a solid support.

### DETAILED DESCRIPTION OF SPECIFIC EMBODIMENTS OF THE INVENTION

The invention provides methods and compositions for detecting activation states of a plurality of signal transducers of circulating cells of a solid tumor in a specific, multiplex, high-throughput assay. Detection of the activation states of multiple signal transducers of the circulating cells facilitates cancer prognosis and diagnosis, and the design of personalized, targeted therapies.

Circulating cells of a solid tumor include cells that have either metastasized or micrometastasized from a solid tumor or cells that are migrating to the tumor (e.g. due to chemoattraction), such as endothelial progenitor cells, circulating endothelial cells, circulating pro-angiogenic myeloid cells, dendritic cells, etc. Patient samples containing the cells can be obtained from any accessible biological fluid (e.g. blood, urine, nipple aspirate, lymph, saliva, fine needle aspirates, etc.). The circulating cells are typically isolated from a patient sample using one or more separation methods such as immunomagnetic separation (e.g. Racila et al, PNAS USA. (1998) 95:4589-94; Bilkenroth et al, Int J Cancer. (2001) 92:577-82; CellTrack™ System by Immunicon (Huntingdon Valley, Pa.)), microfluidic separation (e.g. Mohamed et al, IEEE Trans Nanobioscience. (2004) 3:251-6; Richard Cote, ACS 2006, poster 112), FACS (e.g. Mancuso et al, Blood (2001) 97:3658-61), density gradient centrifugation (e.g. Baker et al, Clin Cancer Res. (2003) 13:4865-71), depletion methods (e.g. Meye et al, Int J Oncol. (2002) 21:521-30), etc.

To preserve the in situ activation states the signal transducers are advantageously extracted shortly after the cells are isolated, preferably within 24, 6, or 1 hr, more preferably within 30, 15 or 5 minutes. The isolated cells may also be advantageously incubated with growth factors usually at nanomolar to micromolar concentrations for about 1-30 minutes, to resuscitate or stimulate signal transducer activation (see e.g. Irish et al, Cell (2004) 118:217-28). Stimulatory growth factors include epidermal growth factor (EGF), heregulin (HRG), TGFα, PIGF, Ang, NRG1, PGF, TNF, VEGF, PDGF, IGF, FGF, HGF, cytokines, etc. To evaluate potential anti-cancer therapies for an individual patient, prior to growth factor stimulation, the isolated cells can be incubated with one or more anti-cancer drugs of varying doses. The differential activation of signaling pathways with and without anti-cancer drugs aids in the selection of a suitable cancer therapy at the proper dose for each individual patent. After isolation, anti-cancer agent treatment, and/or stimulation, the cells are lysed to extract the signal transducers. Preferably the lysis is initiated less than 30 minutes after stimulation, and more preferably less than 15 or 5 minutes after stimulation. Alternatively, the lysate can be stored at −80° C. until use.

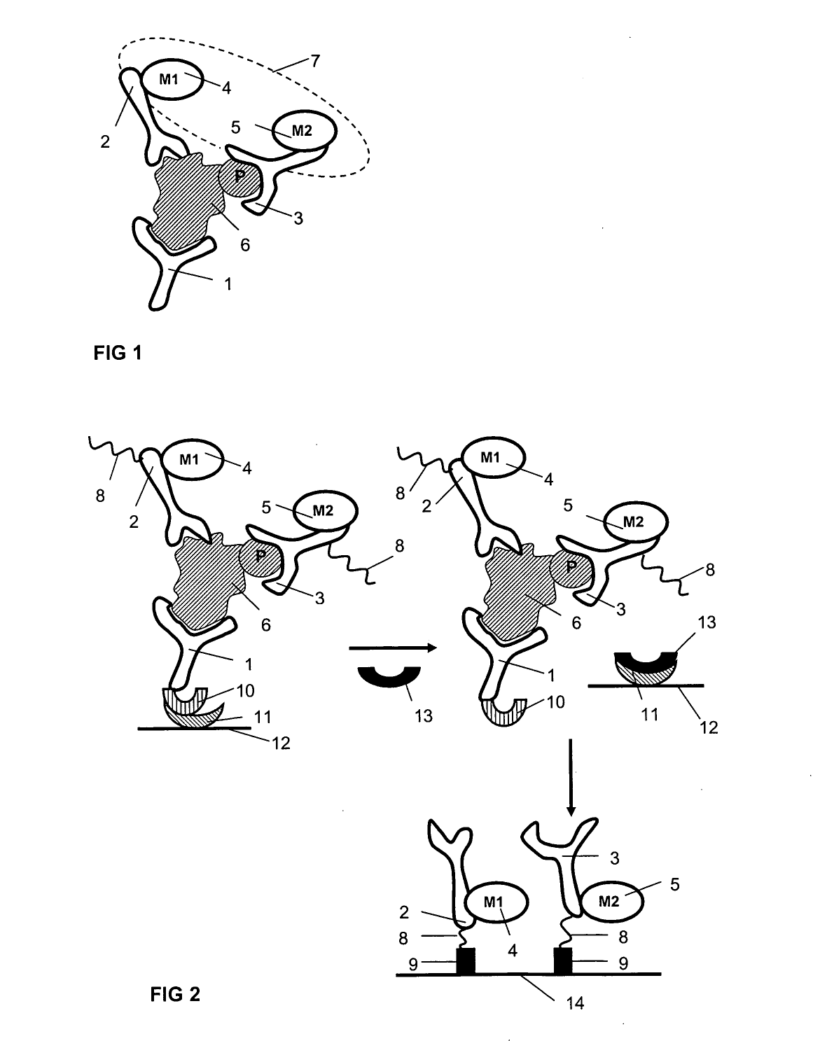

Disclosed methods comprise a contacting step in which the signal transducers extracted from the cells are contacted with first, second, and third binding partners specific for each of the signal transducers. Examples of binding partners include ligands or receptors of the signal transducer, substrates of the signal transducer, binding domains (e.g. PTB, SH2, etc), aptamers, etc. In a preferred embodiment the binding partners are antibodies. Referring to FIG. 1, the first binding partner 1 and the second binding partner 2 each bind the corresponding signal transducer 6 independent of its activation state. The third binding partner 3 binds the signal transducer 6 dependent of its activation state (i.e. the third binding partners will only bind activated signal transducer, which is depicted in FIG. 1 as a phosphorylated residue). The second and third binding partners are labeled with first and second moieties, respectively (designated M1, 4, and M2, 5, in FIG. 1), which enable detection of binding partners that have specifically bound to the signal transducers, as discussed further below. The binding partners are selected to minimize competition between them with respect to signal transducer binding (i.e. all three binding partners can simultaneously bind their corresponding signal transducer as depicted in FIG. 1). Suitable binding partners for numerous signal transducers are commercially available (see e.g. Upstate (Temecula, Calif.), Biosource (Camarillo, Calif.), Cell Signaling Technologies (Danvers, Mass.), R&D Systems (Minneapolis, Minn.), Lab Vision (Fremont, Calif.), Santa Cruz Biotechnology (Santa Cruz, Calif.), Sigma (St. Louis, Mo.), and BD Biosciences (San Jose, Calif.)).

Activation states of interest include phosphorylation, ubiquitination, complexation with another cellular molecule, etc. Signal transducers of particular interest in cancer are well-known; some examples are provided in the following list, with activation states of interest indicated in parentheses: EGFR (EGFRvIII, phosphorylated (p-) EGFR, EGFR:Shc, ubiquitinated (u-) EGFR, and p-EGFRvIII); ErbB2 (p85:truncated (Tr)-ErbB2, p-ErbB2, p85:Tr-p-ErbB2, Her2:Shc, ErbB2:PI3K); ErbB3 (p-ErbB3, ErbB3:PI3K, p-ErbB3:PI3K, ErbB3:Shc); ErbB4 (p-ErbB4, ErbB4:Shc); IGF-1R (p-IGF-1R, IGF-1R:IRS, IRS:PI3K, p-IRS, IGF-1R:PI3K); INSR (p-INSR); KIT (p-KIT); FLT3 (p-FLT3); HGFR1 (p-HGFR1); HGFR2 (p-HGFR2); RET (p-RET); PDGFRα (p-PDGFRα); PDGFRβ (p-PDGFRβ); VEGFR1 (p-VEGFR1, VEGFR1:PLCg, VEGFR1:Src); VEGFR2 (p-VEGFR2, VEGFR2 PLCγ, VEGFR2: Src, VEGFR2:heparin sulphate, VEGFR2:VE-cadherin); VEGFR3 (p-VEGFR3); FGFR1 (p-FGFR1); FGFR2 (p-FGFR2); FGFR3 (p-FGFR3); FGFR4 (p-FGFR4); Tie1 (p-Tie1); Tie2 (p-Tie2); EphA (p-EphA); EphB (p-EphB); NFκB and/or IκB (p-Iκ (S32), p-NFκB (S536), p-P65:IκBa); Akt (p-Akt (T308, S473)); PTEN (p-PTEN); Bad (p-Bad (S112, S136), Bad:14-3-3); mTor (p-mTor (S2448)); p70S6K (p-p70S6K (T229, T389)); Mek (p-Mek (S217, S221)); Erk (p-Erk (T202, Y204)); Rsk-1 (p-Rsk-1 (T357, S363)); Jnk (p-Jnk (T183, Y185)); P38 (p-P38 (T180, Y182)); Stat3 (p-Stat-3 (Y705, S727)); Fak (p-Fak (Y576)); Rb (p-Rb (S249, T252, S780)); Ki67; p53 (p-p53 (S392, S20)); CREB (p-CREB (S133)); c-Jun (p-c-Jun (S63)); cSrc (p-cSrc (Y416)); paxillin (p-paxillin (Y118)).

In the contacting step, the signal transducers extracted from the cells are also contacted with a solid support. The solid support can comprise any material and configuration suitable for performing the disclosed solid phase binding assay (e.g. plastic or glass tubes, beads, slides, or microtiter plates; porous filter paper; magnetic beads; etc.). The solid support has a surface comprising: (a) the first binding partners restrained in an array and adapted to selectively immobilize and organize the corresponding signal transducers on the array; or (b) a plurality of capture molecules restrained in an array, wherein the first binding partners or second and third binding partners comprise capture tags that specifically bind the capture molecules, and the capture molecules are adapted to selectively immobilize and organize the corresponding tagged binding partners on the array. The array can be any configuration that allows discrete signals for each of the activated signal transducers to be detected. For example, the array can be a line or a grid of distinct regions (e.g. dots or spots) on the support surface, where each region contains a different first binding partner or capture molecule (i.e. to bind the capture tags of the first or second and third binding partners). The array is configured for use in methods where the activation states of a plurality of signal transducers are detected in a single, multiplex assay. In various embodiments, the plurality is at least 3, 5, 10, 15, 20, or more.

When the support surface comprises the first binding partners restrained in an array, the contacting step may comprise contacting the extracted signal transducers with the support surface to selectively immobilize and organize the corresponding signal transducers, and then contacting the immobilized signal transducers with one or more “cocktails” that collectively comprise the second and third binding partners of all the signal transducers to be assayed. Higher assay sensitivity can be achieved by solution binding kinetics. Accordingly, in preferred embodiments, the contacting step comprises incubating the extracted signal transducers in solution with the second and third binding partners, and contacting the resulting signal transducer-binding partner complexes (i.e. the second and third binding partners bound to the corresponding signal transducers) with the support surface to selectively immobilize and organize the corresponding signal transducers on the array. The term “incubating” is used synonymously with “contacting” and does not imply any specific time or temperature requirements unless otherwise indicated. After binding complexes of the three binding partners and corresponding signal transducers are formed, the complexes are separated from unbound binding partners, typically by washing the complexes with a suitable wash buffer. Other separation methods can also be used, such as magnetic bead separation, polystyrene beads, etc.

In embodiments where the support surface comprises capture molecules restrained in an array, the contacting step preferably comprises incubating the extracted signal transducers in solution with the first, second, and third binding partners, using an excess of all three binding partners to drive the reaction to completion. In one variation of the method, the resulting signal transducer-binding partner complex (i.e. the first, second, and third binding partners bound to the corresponding signal transducers) is attached to a solid phase and washed to remove unbound binding partners. Referring to FIG. 2, one of the binding partners (typically the first) can comprise a capture moiety 10. The complexes are attached to a solid phase 12 via a capture agent 11 that is adhered to the solid phase and binds the capture moiety, thereby immobilizing the complex. The immobilized complex is washed with a suitable buffer, and then released from the solid phase by the addition of a releasing agent 13. The releasing agent may function by any mechanism that results in the release of the washed complex. In one embodiment, the capture moiety comprises a cleavable site that is recognized and cleaved by the releasing agent. In another embodiment, depicted in FIG. 2, the releasing agent 13 competes with the capture moiety, 10 for binding to the capture agent 11. For example, the capture agent may be a first oligonucleotide that hybridizes with a partially complementary oligonucleotide (i.e. the capture moiety) attached to the first binding partner; and the releasing agent may be an oligonucleotide that is fully complementary to the capture agent, resulting in strand displacement and release of the washed complex from the solid phase. Other examples of suitable capture moiety/capture agent/releasing agents that can be used include: 2,4 dinitrophenol (DNP)/antiDNP antibody/2,4 DNP lysine; T2/antiT3 antibody/T3; ouabain/antiDigoxin antibody/digoxin; dethiobiotin/streptavidin/biotin (see e.g. Ishikawa et al, J. Clinical Lab Analysis (1998) 12:98-107).

After the washed complex is released from the solid phase it is either: 1) contacted with a support surface comprising capture molecules restrained in an array that specifically bind capture tags on the first binding partner, or 2) it is dissociated, and the dissociated second and third binding partners are contacted with a support surface comprising capture tags that specifically bind capture tags on the second and third binding partners. FIG. 2 depicts the embodiment where the washed complex is dissociated and the dissociated second and third binding partners are contacted with the support surface 16. The support surface comprises a plurality of capture molecules restrained in an “addressable”, or “zip code”, array. Each distinct region of the array comprises a unique capture molecule 9 that specifically binds the capture tags 8 that are on the second and third binding partners, thereby restraining and organizing the tagged binding partners in the array. In a preferred embodiment, the capture molecules and capture tags are oligonucleotides that specifically hybridize to each other. Addressable arrays comprising oligonucleotide capture molecules are well-known (see e.g. Keramas et al, Lab Chip (2004) 4:152-8; Delrio-Lafreniere et al, Diagn Microbiol Infect Dis. (2004) 48:23-31).

In another variation of the method, the support surface comprises the capture molecules restrained in the array, and the first binding partners comprise the capture tags. In the contacting step, the extracted signal transducers are incubated in solution with the first, second, and third binding partners to form signal transducer-binding partner complexes. The complexes are contacted with the support surface and immobilized and organized on the array. Prior to the detecting step, the immobilized complexes are washed to remove uncomplexed binding partners, then the washed complexes are sequentially released from the support surface (e.g. by strand displacement, as described above) and the first and second moieties for each of the signal transducers being assayed are detected by a suitable method as described below.

The presence of the first and second moieties of the restrained second and third binding partners at each distinct region of the array is directly or indirectly detected. Examples of moieties that are directly detected include chromophores, colloidal gold, colored latex, fluorophores, etc. In one embodiment, the first and second moieties are first and second fluorophores. Any pair of fluorophores that provide a distinguishable readout while in close proximity to each other can be used such as CY3/CY5, CY5/phycoerthrin, etc. Alternatively, if an oligonucleotide addressable array is used, the first and second moieties can be the same fluorophore delivered to different zip codes. Laser scanning confocal microscopy can be used to detect fluorophore moieties that are adhered on the array. In assays where the signal transducer-binding partner complexes are released from the array prior to detection, such as in strand displacement assays, suitable methods for detecting the fluorophore moieties include capillary flow confocal laser induced fluorescence, nano-HPLC, micro-capillary electrophoresis, etc.

Moieties that are detected indirectly may require an activation step or steps to generate a detectable signal, such as an enzyme moiety that is contacted with a substrate to generate a detectable signal. In a specific embodiment, the first and second moieties generate first and second signals wherein the second signals are detectable and generated dependent on channeling of the first signals between the moieties. Referring to FIG. 1, the second binding partner 2 is labeled with a first moiety (M1) 4; and the third binding partner is labeled with a second moiety (M2) 5. Binding of both the second binding partner 2 and the third binding partner 3 to the signal transducer 6 brings the first moiety 4 within sufficient proximity (depicted by the area inside the dotted line 7) to the second moiety 5 such that a signal generated by the first moiety can channel to the second moiety resulting in the generation of a detectable and/or amplifiable signal. Various methods for signal channeling are known in the art such as FRET, time-resolved fluorescence-FRET, LOCI, etc. An advantage of signal channeling, as used in the methods of the invention, is that a single detectable signal is generated for only those signal transducers that have bound all three binding partners, resulting in increased assay specificity, lower background, and simplified detection.

In one example of signal channeling, the first and second moieties are two enzymes, such as an oxidase and a peroxidase, respectively. When the first enzyme is contacted with a substrate, it generates a first signal that is channeled to the second moiety, which, in the presence of a substrate for the second moiety, generates a detectable second signal or an intermediate, amplifiable signal. For example the first moiety can be glucose oxidase (GO), and the second moiety can be horseradish peroxidase (HRP). Methods of using GO and HRP in a proximity channeling immunoassay have been reported (e.g. Langry and Horn, 1999, US Dept. of Energy Report No. UCRL-ID-136797). If glucose and luminol are added to a GO-labeled second binding partner, the GO converts the glucose to D-glucono-1,5-lactone and generates the first signal, hydrogen peroxide (H₂O₂). If the second binding partner is in channeling proximity to an HRP-labeled third binding partner, the H₂O₂ will channel to the third binding partner and be used by the HRP to oxidize the luminol and produce light (i.e. a detectable second signal). To achieve greater assay sensitivity, the second moiety and substrate can be selected to generate an amplifiable signal that is generated upon channeling of the first signal to the second moiety. For example, instead of adding luminol along with the glucose in the above example, biotinylated tyramide, an amplification reagent, is added. The HRP and generated H₂O₂ oxidize the tyramide to generate a reactive tyramide radical that covalently binds nearby nucleophilic residues. Next, streptavidin labeled HRP is added, which binds the biotinylated tyramide. After a washing step to remove unbound reagents, H₂O₂ and luminol are added generating an amplified second signal (light) which is detected using any suitable method.

In another example of signal channeling, the first moiety is a photosensitizer (e.g. methylene blue, rose bengal, porphyrins, squarate dyes, phthalocyanines, etc.) and the second moiety is a large molecule labeled with multiple haptens that are protected with protecting groups that prevent binding of the haptens to a specific binding partner (e.g. ligand, antibody, etc.). For example, the second moiety may be a dextran molecule labeled with protected biotin, coumarin, or fluorescein molecules. Suitable protecting groups include phenoxy-, analino-, olefin-, thioether-, and selenoether-protecting groups, etc. Suitable photosensitizers and protected hapten molecules for use in oxygen channeling assays are described in U.S. Pat. No. 5,807,675 to Davalian et al. When the photosensitizer is excited with light, it generates singlet oxygen as a first signal. If the second binding partner is within channeling proximity to the labeled third binding partner, the singlet oxygen channels to the third binding partner and reacts with thioethers on the protecting groups to yield carbonyl groups (ketones or aldehydes) and sulphinic acid, releasing the protecting groups from the haptens. The unprotected haptens are then available to specifically bind to a specific binding partner that can generate a detectable signal. For example, where the hapten is biotin, the specific binding partner can be an enzyme-labeled streptavidin. Exemplary enzymes include alkaline phosphatase, β-galactosidase, HRP, etc. After washing to remove unbound reagents, the second signal is generated by adding a detectable (e.g. fluorescent, chemiluminescent, chromogenic, etc) substrate of the enzyme, and detected using suitable methods and instrumentation known in the art.

In another example of oxygen channeling, the first moiety is a photosensitizer and the second moiety is an enzyme-inhibitor complex. The enzyme and inhibitor (e.g. phosphonic acid-labeled dextran) are linked together by a cleavable linker (e.g. thioether). When singlet oxygen is generated and channeled to the second binding partner, it reacts with the thioether, releasing the inhibitor from the enzyme, thereby activating the enzyme. An enzyme substrate is added to generate a detectable second signal, or alternatively, an amplification reagent is added to generate an amplified second signal.

As an alternative to using a photosensitizer in the oxygen channeling assay, the first moiety can be an enzyme such as myeloperoxidase or chloroperoxidase that is activated by H₂O₂ to generate the singlet oxygen.

In a further embodiment of a channeling assay, the first moiety is HRP, and the second moiety comprises protected haptens, or is an enzyme-inhibitor complex as described above, and the protecting groups comprise p-alkoxy phenol. The addition of phenylenediamine and H₂O₂ generates a reactive phenylene diimine which channels to the second moiety and reacts with p-alkoxy phenol protecting groups to yield exposed haptens or a reactive enzyme. The second signal is generated and detected as described above (see e.g. U.S. Pat. No. 5,532,138 to Singh et al, and U.S. Pat. No. 5,445,944 to Ullman et al.).

The invention also provides kits for performing the methods of the invention comprising the first, second, and third binding partners; and the solid support. In one embodiment of the kit, the solid support has a surface comprising: (a) the first binding partners restrained in an array and adapted to selectively immobilize and organize the corresponding signal transducers on the array; or (b) a plurality of capture molecules restrained in an array, wherein the first binding partners comprise capture tags that specifically bind the capture molecules, and the capture molecules are adapted to selectively immobilize and organize the corresponding tagged binding partners on the array; and the first and second moieties are adapted to generate first and second signals wherein the second signal is detectable and generated dependent on channeling of the first signal between the moieties. In another embodiment of the kit, the solid support surface comprises capture molecules restrained in the array, wherein the first binding partners or second and third binding partners comprise capture tags that specifically bind the capture molecules to restrain and organize the binding partners in the array. The kits contain instructions for methods of using the kit to detect the activation states of a plurality of signal transducers of circulating cells of a solid tumor in a specific, multiplex, high-throughput assay. The kit may also contain any of the additional reagents described above with respect to performing specific methods of the invention such as tyramide signal amplification reagents, substrates for enzyme moieties that are on the second and third binding partners, wash buffers, capture/release reagents etc.

### EXAMPLE 1

### Detection of Circulating Tumor Cell (CTC) Signaling in Breast Cancer Patients

Microarray fabrication and processing are adapted from methods described by Chan et al. (Nat Med. (2004) 10:1390-6). Antibodies (1 mg/ml) against the signal transducers EGFR, ErbB2, ErbB3, ErbB4, IGF-1R, Akt, Erk, p70S6K, Bad, Rsk, Mek, cSrc, Cytokeratin, Tubulin, beta actin, and Anti-Mouse Antibody (Positive Control) (4/4 array) are transferred to a 384-well polypropylene plate (50 μls/well) using a contact printing robotic microarrayer (Bio-Rad) fitted with solid spotting pins to spot antibodies onto Fast slides (Schleicher and Schuell Biosciences). Slides coated with 8 sectored pads are used. After printing, the slides are blocked with 3% casein solution. Slides are stored at least overnight under dry conditions before use.

Patient selection criteria, sample preparation methods, and study design are adapted from published studies evaluating circulating tumor cells in women with suspected breast cancer (see Wulfing et al, Clin Cancer Res. (2006) 12):1715-20; and Reinholz et al, Clin Cancer Res. (2005) 11:3722-32). Women with a breast abnormality detected on imaging and who are to undergo a breast biopsy are approached for this study. At least forty-two patients who are diagnosed with primary breast cancer are included. At least thirty-five patients have no sign of overt metastasis at the time of primary diagnosis. At least seven patients have distant metastases at diagnosis and are considered a positive reference group. None of the patients have a history of previous cancer. Age and treatment information (i.e. surgical therapy, chemotherapy, radiotherapy, endocrine therapy, etc.) is collected for each patient. Approximately 20 mL of blood is collected from each patient and all blood samples are assigned a unique identification number. All assays are done with the investigators blinded to the results of the biopsy.

Peripheral blood (18 mL) is added to an Accuspin Histopaque-1077 system (Sigma Aldrich, St. Louis, Mo.) and centrifuged at 1,500 rpm for 10 minutes in a Beckman CS-6R tabletop centrifuge (Beckman Instruments, Palo Alto, Calif.). The mononuclear cell layer is removed, washed twice with PBS, and diluted to 1 nL with PBS/0.1% bovine serum albumin. The epithelial cells are enriched by immunomagnetic capture using antibodies against Ber-EP4 attached to magnetic beads using the Dynabeads Epithelial Enrich kit according to the manufacturer's instructions (Dynal). The cells are mixed with 1-10⁷ beads in a volume of 20 mL while rocking for 1 hour. The Ber-EP4 antibody recognizes two glycoproteins on the surface and in the cytoplasm of epithelial cells except the superficial layers of squamous epithelial cells, hepatocytes, and parietal cells. The suspension is placed on a magnet for at least 6 minutes and the supernatant is carefully removed. The cells attached to the magnetic beads are washed thrice with 1 nL PBS/0.1% bovine serum albumin. Growth factors TGFα (100 nM), Hrg (100 nM) IGF (100 nM) are added to the cells and incubated at 37° C. for 5 minutes. The cells are concentrated and lysed with the lysis binding buffer supplied with the kit. The lysed cell suspension (with beads attached) is stored at 80° C. until processing.

Assay Method 1: Lysates (40 μl) are applied to the array, incubated overnight, and washed three times with wash buffer (from Schleicher and Schuell Biosciences). HRP-labeled anti-phospho antibody (conjugated as described in Kuhlmann W D (1984) Immuno enzyme techniques, Verlag Chemie, weinheim, pp 1-162) against each of the signal transducers and anti-total antibody (i.e. activation state-independent) labeled with glucose oxidase (conjugated as described in Kuhlmann W D, supra) are added to the array, incubated for 2 hours, and washed three times with wash buffer. Tyramide reagent (Molecular Probes) and glucose are added and the reaction is developed for one hour then washed three times. The array is incubated with streptavidin-HRP for 30 minutes, and washed and developed using enhanced luminol (Molecular Probes). Signal is detected using a CCD camera.

Assay Method 2: Antibodies (1 mg/ml) against 2,4 dinitrophenol (DNP) are transferred to a 384-well polypropylene plate (50 μl/well) using a contact printing robotic microarrayer (Bio-Rad) fitted with solid spotting pins to spot antibodies onto Fast slides (Schleicher and Schuell Biosciences). Slides coated with 8 sectored pads are used. After printing, the slides are blocked with 3% casein solution. Slides are stored at least overnight under dry conditions before use.

The lysate (40 μl) is mixed with total 2,4 DNP-labeled antibody against each of the above signal transducers, anti-phospho antibody labeled with Oligo and Alexa 647, and anti-total antibody labeled with Oligo and Alexa 647, added to the anti-DNP antibodies, incubated for overnight, and washed three times with wash buffer. 2,4 Dinitro Lysine (Molecular Probes) is added to release the immune-complexes from the anti-2,4 DNP antibodies. The released immune-complexes are added to a zip-code array (see e.g. Keramas et al., Lab Chip (2004) 4:152-8; Delrio-Lafreniere et al, Diagn. Microbiol. Infect. Dis (2004) 48:23-31) and incubated overnight. The array is washed three times and the processed slides are scanned using GenePix 4000A microarray scanner (Axon Scanner) at 10 micron resolution.

### EXAMPLE 2

### Detection of Circulating Endothelial Cell (CEC) and Circulating Endothelial Precursor Cell (CEP) Signaling in Breast Cancer Patients

The same patient samples, sample preparation, and assay methods described in Example 1 are used, except that CEC and CEP cells are enriched by immunomagnetic capture using the monoclonal antibody, PH1H2 or CD146, attached to magnetic Dynabeads. A microarray is fabricated and processed using the methods described in Example 1, except that the arrayed antibodies are against the following: VEGFR1, VEGFR2, VEGFR3, TIE 1, TIE 2, PDGFR-α, PDGFR-β, FGFR1, FGFR2, Akt, Erk p70S6K, Rsk, cSrc, and beta actin. The assay methods described in Example 1 are used to detect CEC and CEP signaling.

What is claimed is:

1. A method for detecting activation states of a plurality of signal transducers of circulating cells of a solid tumor in a specific, multiplex, high-throughput assay, the method comprising steps:
contacting the signal transducers extracted from the cells with first, second, and third binding partners specific for each of the signal transducers, and a solid support having a surface comprising:
(a) the first binding partners restrained in an array and adapted to selectively immobilize and organize the corresponding signal transducers on the array; or
(b) a plurality of capture molecules restrained in an array, wherein the first binding partners comprise capture tags that specifically bind the capture molecules, and the capture molecules are adapted to selectively immobilize and organize the corresponding tagged binding partners on the array,
whereby the first, second, and third binding partners specifically bind to and restrain the corresponding signal transducers on the array,
wherein the second binding partners bind the corresponding signal transducers independent of their activation state and are labeled with a first moiety,
the third binding partners bind the corresponding signal transducers dependent of their activation state and are labeled with a second moiety, and
the first and second moieties generate first and second signals wherein the second signal is detectable and generated dependent on channeling of the first signal between the moieties; and
detecting the generated second signal as an indication of the activation states of the plurality of signal transducers.

2. The method of claim 1 wherein prior to the contacting step, the cells are isolated from a patient sample by immunomagnetic separation.

3. The method of claim 1 wherein prior to the contacting step, the cells are isolated from a patient sample and stimulated in vitro with growth factors to activate the signal transducers.

4. The method of claim 1 wherein the first, second, and third binding partners are antibodies.

5. The method of claim 1 wherein the support surface comprises the first binding partners restrained in the array, and the contacting step comprises:
contacting the signal transducers with the support surface to selectively immobilize and organize the corresponding signal transducers on the array; and
contacting the immobilized signal transducers with the second and third binding partners to bind the second and third binding partners to the corresponding signal transducers.

6. The method of claim 1 wherein the support surface comprises the first binding partners restrained in the array, and the contacting step comprises:
incubating the signal transducers in solution with the second and third binding partners to specifically bind the corresponding signal transducers to the second and third binding partners and form signal transducer-binding partner complexes; and
contacting the complexes with the support surface to immobilize and organize the corresponding signal transducers on the array.

7. The method of claim 1 wherein the support surface comprises the capture molecules restrained in the array, and the contacting step comprises:
incubating the signal transducers in solution with the first, second, and third binding partners to specifically bind the binding partners to the corresponding signal transducers and form signal transducer-binding partner complexes; and
contacting the complexes with the support surface to immobilize and organize the corresponding tagged binding partners on the array.

8. The method of claim 1 wherein the first moiety is an oxidase, the first signal is H₂O₂, and the second moiety is a peroxidase, and the method further comprises after the contacting step:
applying an oxidase substrate and biotin-tyramide to the support surface, wherein the substrate and oxidase generate the H₂O₂ which channels to and reacts with the peroxidase and the biotin-tyramide to produce activated biotin-tyramide which binds the restrained signal transducers;
contacting the support surface with streptavidin-horseradish peroxidase (HRP) to bind the streptavidin-HRP to the signal transducer-bound biotin-tyramide;
removing unbound streptavidin-HRP from the support surface; and
contacting the bound streptavidin-HRP with an HRP substrate and additional H₂O₂ to generate the second signal.

9. The method of claim 1 wherein the first moiety is a photosensitizer, the first signal is singlet oxygen, the second moiety comprises a hapten protected by a protecting group, and the method further comprises after the contacting step:
exposing the solid support to light, whereby the photosensitizer generates the singlet oxygen which channels to the hapten and reacts with the protecting group to release the protecting group from the hapten;
incubating the unprotected hapten with an enzyme-labeled hapten binding partner to bind the labeled hapten binding partner to the unprotected hapten;
removing unbound labeled hapten binding partner from the support surface; and
contacting the support surface with a substrate of the enzyme to generate the second signal.

10. The method of claim 1 wherein the first moiety is a photosensitizer, the first signal is singlet oxygen, the second moiety comprises an enzyme inactivated by thioether linkage to an enzyme inhibitor, and the method further comprises after the contacting step:
exposing the solid support to light, whereby the photosensitizer generates the singlet oxygen which channels to the enzyme and reacts with the thioether linkage to release the enzyme from the inhibitor and activate the enzyme; and
contacting the support surface with a substrate of the activated enzyme to generate the second signal.

11. A kit for performing the method of claim 1 comprising:
the first, second, and third binding partners; and
the solid support.

12. A method for detecting activation states of a plurality of signal transducers of circulating cells of a solid tumor in a specific, multiplex, high-throughput assay, the method comprising steps:
incubating the signal transducers extracted from the cells in solution with optional first, and with second and third binding partners specific for each of the signal transducers to specifically bind the corresponding signal transducers to the second and third binding partners;
contacting the second and third binding partners that have specifically bound to the corresponding signal transducers with a solid support having a surface comprising:
(a) the first binding partners specific for each of the signal transducers restrained in an array and adapted to selectively immobilize and organize the corresponding signal transducers on the array; or
(b) a plurality of capture molecules restrained in an array, wherein the first or the second and third binding partners comprise capture tags that specifically bind the capture molecules, and the capture molecules are adapted to selectively immobilize and organize the corresponding tagged binding partners on the array,
whereby the second and third binding partners are restrained on the support surface,
wherein prior to, or concurrently with, being restrained on the support surface the second and third binding partners form signal transducer-binding partner complexes with the corresponding signal transducers and first binding partners,
the second binding partners bind the corresponding signal transducers independent of their activation state and are labeled with a first moiety, and
the third binding partners bind the corresponding signal transducers dependent of their activation state and are labeled with a second moiety; and
detecting the first and second moieties of the restrained second and third binding partners as an indication of the activation states of the plurality of signal transducers.

13. The method of claim 12 wherein prior to the contacting step, the cells are isolated from a patient sample by immunomagnetic separation.

14. The method of claim 12 wherein prior to the contacting step, the cells are isolated from a patient sample and stimulated in vitro with growth factors to activate the signal transducers.

15. The method of claim 12 wherein the first, second, and third binding partners are antibodies.

16. The method of claim 12 wherein the support surface comprises the first binding partners restrained in the array, and the contacting step comprises:
incubating the signal transducers extracted from the cells in solution with the second and third binding partners to specifically bind the corresponding signal transducers to the second and third binding partners; and
contacting the resultant second and third binding partner-bound signal transducers with the support surface to form, immobilize, and organize the complexes on the array.

17. The method of claim 12 wherein the support surface comprises the capture molecules restrained in the array, the first binding partners comprise the capture tags, and the contacting step comprises:
incubating the signal transducers extracted from the cells in solution with the first, second, and third binding partners to form the complexes; and
contacting the complexes with the support surface to immobilize and organize the complexes on the array, wherein prior to the detecting step the immobilized complexes are washed to remove uncomplexed binding partners, and wherein the detection step comprises:
releasing the washed complexes from the support surface and detecting the first and second moieties by a method selected from the group consisting of capillary flow confocal laser induced fluorescence, nano-HPLC, and micro-capillary electrophoresis.

18. The method of claim 12 wherein the support surface comprises the first binding partners restrained in the array, the first and second moieties are fluorophores, and the detection step comprises laser scanning confocal microscopy.

19. The method of claim 12 wherein the support surface comprises the capture molecules restrained in the array, the second and third binding partners comprise the capture tags, and the contacting step comprises:
incubating the signal transducers extracted from the cells in solution with the first, second, and third binding partners to form the complexes;
attaching the complexes to a solid phase and washing the attached complexes to remove uncomplexed binding partners;
releasing the washed complexes from the solid phase;
dissociating the released complexes to dissociate the second and third binding partners from the complexes; and
contacting the dissociated second and third binding partners with the support surface, wherein the capture tags specifically bind the capture molecules to restrain and organize the tagged binding partners on the array.

20. A kit for performing the method of claim 12 comprising:
the first, second, and third binding partners; and
the solid support, wherein the support surface comprises the capture molecules restrained in the array, wherein the first binding partners or second and third binding partners comprise the capture tags that specifically bind the capture molecules to restrain and organize the binding partners in the array.
